(19) United States
(12) Patent Application Publication
(10) Pub. No.: US 2017/0035011 A1
(43) Pub. Date: Feb. 9, 2017

(54) METHODS OF IMPROVING GERMINATION OF PLANT EMBRYOS

(71) Applicant: WEYERHAEUSER NR COMPANY, Federal Way, WA (US)

(72) Inventors: James A. Grob, Bonney Lake, WA (US); Pramod K. Gupta, Federal Way, WA (US); Patrick M. Brownell, Tacoma, WA (US); Anthony P. Swanda, Snoqualmie, WA (US)

(21) Appl. No.: 15/228,891

(22) Filed: Aug. 4, 2016

Related U.S. Application Data
(60) Provisional application No. 62/201,851, filed on Aug. 6, 2015.

(51) Int. Cl. A01H 4/00; C12N 5/00

(52) U.S. Cl. CPC A01H 4/00; C12N 5/0025; C12N 2500/34

(57) ABSTRACT

A method of improving germination of plant embryos is provided. The method entails treating a culture of plant tissue with a liquid multiplication medium comprising a plant non-metabolizable sugar. Also provided is a multiplication medium for liquid cultures of plant cells comprising isomaltulose.

### CROSS-REFERENCE TO RELATED APPLICATION

This application benefits from the priority claim to U.S. Provisional Patent Application No. 62/201,851, filed on Aug. 6, 2015, the content of which is incorporated herein by reference in its entirety.

### TECHNICAL FIELD

The technical field includes methods of improving germination of plant embryos.

### BACKGROUND

Modern silviculture often requires the planting of large numbers of genetically identical plants that have been selected to have advantageous properties. Production of new plants by sexual reproduction, which yields botanic seeds, is usually not feasible. Asexual propagation, via the culturing of somatic or zygotic embryos, has been shown for some species to yield large numbers of genetically identical embryos, each having the capacity to develop into a normal plant.

Somatic cloning is the process of creating genetically identical plants from plant tissue other than male and female gametes. In one approach to somatic cloning, plant tissue is cultured in an initiation medium that includes hormones, such as auxins and/or cytokinins, to initiate formation of embryogenic tissue, such as embryogenic suspensor masses, that are capable of developing into somatic embryos. Embryogenic suspensor mass, (ESM), has the appearance of a whitish translucent mucilaginous mass and contains early stage embryos. The embryogenic tissue is further cultured in a multiplication medium that promotes multiplication and mass production of the embryogenic tissue. The embryogenic tissue is then cultured in a development medium that promotes development and maturation of cotyledonary somatic embryos that can, for example, be placed on germination medium to produce germinants.

Embryo germination remains a common and particularly challenging issue. There remains a need for generating improved methods that are useful for improving rates of germination conversion of somatic embryos to provide a large number of normal germinants.

### SUMMARY

Provided herein include methods of improving germination of plant embryos, comprising treating a culture of plant tissue with a liquid multiplication medium comprising a non-metabolizable sugar or semi non-metabolizable sugar.

### BRIEF DESCRIPTION OF THE DRAWINGS

FIG. 1 shows an example of Brix time series for a Genotype. Each treatment is shown in a panel.

FIG. 2 shows an example of Brix time series for a Genotype. Each treatment is shown in a panel

FIG. 3 shows an example of Brix time series for a Genotype. Each treatment is shown in a panel.

FIG. 4 shows an example of Brix time series for a Genotype. Each treatment is shown in a panel.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, “plant non-metabolizable sugar” and “plant semi non-metabolizable sugar” refer to a sugar that is not easily enzymatically broken down by a plant to an extent it can be used as a sole carbon source.

As used herein the “plant non-metabolizable sugar” may be selected from the group consisting of turanose, isomaltulose, lactulose, 3α-galactobiose, lactitol, lactose, 43-galactobiose, palatinitol, and melibiose.

As used herein, “a plant somatic embryo” refers to an embryo produced by culturing totipotent plant cells such as meristematic tissue under laboratory conditions in which the cells comprising the tissue are separated from one another and urged to develop into minute complete embryos. Alternatively, somatic embryos can be produced by inducing “cleavage polyembryogeny” of zygotic embryos. Methods for producing plant somatic embryos suitable for use in the methods of the description are standard in the art and have been previously described. For example, plant tissue may be cultured in an initiation medium that includes hormones to initiate the formation of embryogenic cells, such as embryonic suspensor masses that are capable of developing into somatic embryos. The embryogenic cells may then be further cultured in a multiplication medium that promotes establishment and multiplication of the embryogenic cells. Subsequently, the multiplied embryogenic cells may be cultured in a development medium that promotes the development of somatic embryos, which may further be subjected to post-development treatments such as cold treatments.

As used herein, the term “germination” refers to a physiological process that results in the elongation and growth of a plant embryo organs (root, hypocotyls, cotyledons) and initiation of leaves (epicotyl by the shoot apex located at the base of the cotyledons) after 1 week in the dark followed by 6 weeks in a light room at room temperature.

The term “root” refers to the part of a plant embryo that develops into the primary root of the resulting plant below the hypocotyl.

The term “cotyledon” refers generally to the first whorl of leaf-like structures present on the plant embryo that function primarily as food storage and initial photosynthetic structures.

The term “hypocotyl” refers to the portion of a plant embryo or seedling located below the cotyledons but above the radical.

The term “epicotyl” refers to the portion of the seedling stem (including leaves) that are attached above the cotyledons.

As used herein, the term “category 1” germination is defined by presence of a minimum of a 3 mm root, and a tuft of at least 5 epicotyl leaves, each approximately 5 mm long.

“Category 2” germination is defined as for category 1, but where epicotyls leaf presence is observed, but not meeting the 5 leaves by 5 mm length specification.

Several parameters may be measured to determine the quality of the germinants, such as root, hypocotyl, longest cotyledon leaf length, and epicotyls leaf length.

What is described are methods of improving germination of plant embryos, comprising treating a culture of plant tissue with a liquid multiplication medium comprising a non-metabolizable sugar or semi non-metabolizable sugar. In some embodiments, the liquid multiplication medium contains one or more non-metabolizable sugar or semi non-metabolizable sugar only and no metabolizable sugar. In other embodiments, the liquid multiplication medium contains one or more metabolizable sugar in addition to one or more non-metabolizable sugar or semi non-metabolizable sugar. The ratio of the non-metabolizable or semi non-metabolizable sugar to the metabolizable sugar is, for example, 1:1, 2:1, 3:1, 4:1, 5:1, or 6:1.

In one embodiment, the non-metabolizable sugar or semi non-metabolizable sugar is isomaltulose. Another embodiment further comprises adding a metabolizable sugar, maltose in addition to isomaltulose. The non-metabolizable sugar preferably is at a concentration of at least 1 g/l or at most 40 g/l. In some embodiments, the non-metabolizable sugar is at a concentration of at least 1 g/l, at least 2 g/l, at least 3 g/l, at least 4 g/l, at least 5 g/l, at least 6 g/l, at least 7 g/l, at least 8 g/l, at least 9 g/l, at least 10 g/l, at least 11 g/l, at least 12 g/l, at least 13 g/l, at least 14 g/l, at least 15 g/l, at least 16 g/l, at least 17 g/l, at least 18 g/l, at least 19 g/l, at least 20 g/l, at least 21 g/l, at least 22 g/l, at least 23 g/l, at least 24 g/l, at least 25 g/l, at least 26 g/l, at least 27 g/l, at least 28 g/l, at least 29 g/l, at least 30 g/l, at least 31 g/l, at least 32 g/l, at least 33 g/l, at least 34 g/l, at least 35 g/l, at least 36 g/l, at least 37 g/l, at least 38 g/l, at least 39 g/l. In some embodiments, the non-metabolizable sugar is at a concentration of less than 40 g/l, less than 39 g/l, less than 38 g/l, less than 37 g/l, less than 36 g/l, less than 35 g/l, less than 34 g/l, less than 33 g/l, less than 32 g/l, less than 31 g/l, less than 30 g/l, less than 29 g/l, less than 28 g/l, less than 27 g/l, less than 26 g/l, less than 25 g/l, less than 24 g/l, less than 23 g/l, less than 22 g/l, less than 21 g/l, less than 20 g/l, less than 19 g/l, less than 18 g/l, less than 17 g/l, less than 16 g/l, less than 15 g/l, less than 14 g/l, less than 13 g/l, less than 12 g/l, less than 11 g/l, less than 10 g/l, less than 9 g/l, less than 8 g/l, less than 7 g/l, less than 6 g/l, less than 5 g/l, less than 4 g/l, less than 3 g/l, less than 2 g/l.

The plant non-metabolizable sugar may be one or more of the following sugars: turanose, isomaltulose, lactulose, 3a-galactobiose, lactitol, lactose, galactobiose, palatinitol, and melibiose. The maltose preferably is at a concentration of at least 1 g/l or at most 40 g/l.

The multiplication media may comprise a plant non-metabolizable sugar as an osmotic agent of the multiplication media.

The plant embryo may comprise a conifer plant embryo. The conifer plant embryo may comprise a loblolly pine embryo.

As used herein, the term “embryogenic tissue” refers to any tissue, derived from a conifer, which is capable of producing one or more conifer cotyledonary somatic embryos when treated in accordance with the methods of the disclosure. Thus, the term “embryogenic tissue” includes, for example, conifer embryonal suspensor masses.

Unless stated otherwise, all concentration values that are expressed as percentages are weight per volume percentages.

An example of embryogenic tissue useful in the practice of the present subject matter is embryonal suspensor masses (ESMs). ESMs can be prepared from precotyledonary embryos removed from conifer seed. The seed are typically surface sterilized before removing the precotyledonary embryos, which are then cultured on, or in, a medium that permits formation of ESMs that include early stage embryos in the process of multiplication by budding and cleavage. The medium may, if desired, include hormones that stimulate multiplication of the early stage embryos. Examples of hormones that can be included in the medium are auxins (e.g., 2,4-dichlorophenoxyacetic acid (2,4-D)) and cytokinins (e.g., 6-benZylaminopurine (BAP)). Auxins can be utilized, for example, at a concentration of from 1 mg/L to 200 mg/L. Cytokinins can be utilized, for example, at a concentration of from 0.1 mg/L to 50 mg/L.

The somatic embryogenesis process is a process to develop plant embryos in vitro. Methods for producing plant somatic embryos are known in the art and have been previously described (see, e.g., U.S. Pat. Nos. 4,957,866; 5,034,326; 5,036,007; 5,041,382; 5,236,841; 5,294,549; 5,482,857; 5,563,061; and 5,821,126). Generally, the somatic embryo genesis process includes the steps of (1) initiation, sometimes referred to as induction, to initiate formation of embryo genetic tissue, such as embryogenic suspensor mass (ESM), which is US 2013/0078722 A1 a white mucilaginous mass that includes early stage embryos having a long, thin-walled suspensor associated with a small head with dense cytoplasm and large nuclei; (2) multiplication, sometimes referred to as multiplication, to multiply and mass produce the embryogenic tissue; (3) development, to develop and form mature cotyledonary somatic embryos; and (4) post development steps such as singulation, stratification, germination, placement into manufactured seeds, and transferring to soil for further growth and development.

What is also described is a method for improving germination of a plant zygotic or somatic embryo, comprising: culturing the plant embryo in a multiplication medium comprising a plant non-metabolizable sugar. The method may comprise a loblolly pine ESM. The method may use a plant non-metabolizable sugar at multiplication stage to improve embryo germination. The plant non-metabolizable sugar is preferably used at a concentration of at least 1 g/l and at most 40 g/l. The plant non metabolizable sugar may be one or more of the following sugars: turanose, isomaltulose, lactulose, 3a-galactobiose, lactitol, lactose, 41-galactobiose, palatinitol, and melibiose.

Non-plant-metabolizable sugars have been identified, including turanose (3-O-d-glucopyranosyl-D-fructopyranose), isomaltulose (PALATINOSE™) (6-O-α-d-glucopyranosyl-D-fructose), lactulose (4-O-p-D-galactopyranosyl-p-D-fructofuranose), 3a-galactobiose (3-O-a-D-galactopyranosyl-D-galactopyranose), lactitol (4-O-a-D-galactopyranosyl-D-glucitol), lactose (4-O-(3-D-galactopyranosyl-D-glucose), 40-galactobiose (4-O-p-D-galactopyranosyl-D-galactopyranose), palatinitol (a 1:1 mixture of 6-O-a-D-glucopyranosyl-D-glucitol and 6-O-a-D-glucopyranosyl-D-mannitol), and melibiose (6-a-D-galactopyranosyl-D-glucopyranose), having structures shown below.

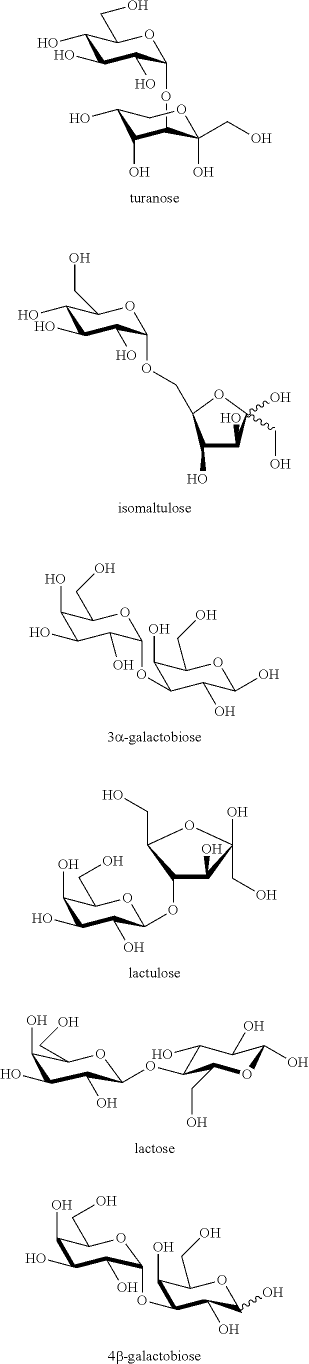

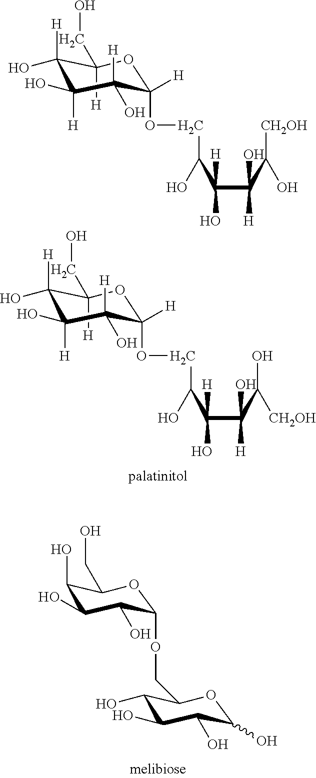

The multiplication media may also contain hormones. Suitable hormones include, but are not limited to, abscisic acid, cytokinins, auxins, and gibberellins. Abscisic acid is a sesquiterpenoid plant hormone that is implicated in a variety of plant physiological processes (see, e.g., Milborrow, J. Exp. Botany 52:1145-1164 (2001); Leung & Giraudat, Ann. Rev. Plant Physiol. Plant Mol. Biol. 49:199-123 (1998)). Auxins are plant growth hormones that promote cell division and growth. Examples of auxins for use in the germination medium include, but are not limited to, 2,4-dichlorophenoxyacetic acid, indole-3-acetic acid, indole-3-butyric acid, naphthalene acetic acid, and chlorogenic acid. Cytokinins are plant growth hormones that affect the organization of dividing cells. Examples of cytokinins for use in the germination medium include, but are not limited to, e.g., 6-benzylaminopurine, 6-furfurylaminopurine, dihydrozeatin, zeatin, kinetin, and zeatin riboside. Gibberellins are a class of diterpenoid plant hormones (see, e.g., Krishnamoorthy (1975) Gibberellins and Plant Growth, John Wiley & Sons). Representative examples of gibberellins useful in the practice of the present description include gibberellic acid, gibberellin 3, gibberellin 4, and gibberellin 7. An example of a useful mixture of gibberellins is a mixture of gibberellin 4 and gibberellin 7 (referred to as gibberellin 4/7), such as the gibberellin 4/7 sold by Abbott Laboratories, Chicago, Ill. When abscisic acid is present in the modified nutritive medium, it is typically used at a concentration in the range of from about 1 mg/l to about 200 mg/l. When present in the nutritive medium, the concentration of gibberellin(s) is typically between about 0.1 mg/l and about 500 mg/l. Auxins may be used, for example, at a concentration of from 0.1 mg/l to 200 mg/l. Cytokinins may be used, for example, at a concentration of from 0.1 mg/l to 100 mg/l.

While not intending to be bound by any particular mechanism of operation, in humans palatinose is of interest as a low glycemic sugar which is only broken down slowly in the small intestine. Palatinose is a non-metabolizable or semi-non metabolizable sugar in plants. As such it can be used as an osmotic agent that cannot be broken down or used by plant cells. Other such sugar moieties include turanose and fluoro suc. Plant studies have shown that palatinose can 1) be perceived as a microbi produced sugar resulting in pathogen defense signaling properties, or 2) may act in various other sugar signaling roles. Lastly, palatinose may also reduce other disaccharide breakdown by competing for enzyme sites.

In loblolly pine SE studies, palatinose was initially used as a non-metabolizable sugar for supplementing osmotic level. In development stage studies containing palatinose with standard maltose and glucose media, palatinose inhibited embryo formation. When used as a part of liquid multiplication media its effect depended on the sugar moieties it was associated with, and its ratio with those moieties. When paired with maltose alone, growth was largely or completely halted depending on sugar ratio, whereas when used with glucose, growth was like control (maltose only). Though the multi-week cessation of multiplication growth was suspected to be lethal, instead abundant embryo formation was found upon plating, and elevated germination in the resulting embryos where palatinose significantly increased the germination percentage of resulting embryos from 4.4% (standard medium) to 9.1%

While not intending to be bound by any particular mechanism of operation, based on current data, it is thought that the role palatinose is playing is one of interfering or saturating disaccharide cleaving enzymes such as invertase or maltase. For example, work done where growth is caused to cease by low temperature, did not have the same germination promoting effect (data not shown). Other ways to achieve growth cessation, under otherwise ideal conditions, by other carbon metabolism alterations remains a possibility.

Substituting the majority of maltose sugar in loblolly pine multiplication medium with palatinose sugar has significantly increased the germination percentage of resulting embryos. Standard medium contains 3.0% maltose, but a medium with 0.6% maltose and 2.4% palatinose increased the category 1 germination percentage from 4.4% (standard medium) to 9.1% in propagation of high value tree experiments, while a medium with 1.0% maltose and 2.0% palatinose raised the category 1 germination percent to 6.8%. A similar and significant improvement was observed in category 1+2 germination percentage, where standard medium germinated at 15.1%, while the media with 2.4% and 2.0% palatinose germinated at 22.4% and 21.8%, respectively. These results are for combining the outcomes of 3, 5, and 7 weeks of treatment.

The effects were observed by the medium with 0.6% maltose+2.4% palatinose immediately arresting all ESM multiplication in multiplication media. The relative concentrations of maltose+palatinose are believed important as 1.0% maltose+2.0% palatinose did not completely arrest ESM multiplication in multiplication media. In experiments, this treatment increased biomass 1.4× over a week's time, while the standard medium increased biomass 3.8× over a week's time. The hypothesis was further verified in experiments where medium containing 0.3% glucose+2.4% palatinose grew at a rate similar to standard medium, while 0.6% maltose+2.4% palatinose did not grow.

At the concentrations considered, including palatinose, multiplication medium is not a variable treatment as the object of multiplication medium is to multiply ESM to a volume sufficient for producing large numbers of embryos. However, it is a possible “pre-treatment”, (i.e., a short-term treatment after multiplication media but before the development stage with short-term being 1 to 2 weeks of treatment). Other experiments suggest that the benefit of palatinose may be time dependent, and that using 0.6% maltose+2.4% palatinose as a “pretreatment” does not result in a substantial germination improvement.

The multiplication medium is formulated to promote the growth and multiplication of conifer embryogenic tissue, such as embryonal suspensor masses. The multiplication medium may be a solid medium, or it may be a liquid medium which, for example, can be agitated to promote growth and multiplication of the embryogenic tissue. The osmolality of the multiplication medium is typically higher than the osmolality of the initiation medium, typically in the range of 180-400 mM/kg. The multiplication medium may contain nutrients that sustain the embryogenic tissue, and may include hormones, such as one or more auxins and/or cytokinins, that promote cell division and growth of the embryogenic tissue. Typically, the concentration of hormones in the multiplication medium is lower than their concentration in the initiation medium. It is generally desirable, though not essential, to include maltose as the sole, or principal, metabolizable sugar source in the multiplication medium. Examples of useful maltose concentrations are within the range of from about 2.5% to about 6.0%.

### Example 1

### Materials and Methods

This example shows representative compositions of media of the present disclosure. This example also shows the compositions of media used in the examples that follow. Table 1 shows Basic Multiplication Media (BM).

TABLE 1
BM

| Media Component | Initial Concentration (mg/L) |
|---|---|
| SALTS | 150 |
| NH₄NO₃ | 909.9 |
| KNO3 | 236.25 |
| Ca(NO₃)₂—4H₂O | 246.5 |
| MgSO₄—7H₂O | 256.5 |
| MgCl₂—6H₂O | 50 |
| KH₂PO₄ | 136 |
| CaCl₂—2H₂O | 50 |
| KI | 4.15 |
| H₃BO₃ | 15.5 |
| MnSO₄—H₂O | 10.5 |
| ZnSO₄—7H₂O | 14.4 |
| Na₂MoO4—H₂O | 0.125 |
| CuSO₄—5H₂O | 0.125 |
| CoCL₂—6H₂O | 0.125 |
| FeSO₄—7H₂O | 28.78 |
| Na₂EDTA | 37.26 |
| Vitamins Amino Acids | |
| Nicotinic Acid | 0.5 |
| Pyridoxine HCl | 0.5 |
| Thymine HCl | 1 |
| Glycine | 2 |
| Solutes | |
| Myo-Inositol | 200 |
| Casamino Acids | 500 |
| L-glutamine | 1000 |
| Maltose | 30000 |
| Additional Components | |
| 2,4D (2,4dichlorophenoxyacetic acid | 3.0 |
| Kinetin (6-fur-furylaminopurine | 0.25 |
| BAP (6-benzylaminopurine) | 0.25 |
| Gellan Gum | 1600 |

Table 2 shows components added to BM to make Development Media (DM).

TABLE 2
DM

| Component | Initial Concentration (mg/L) |
|---|---|
| L-Proline | 100 |
| L-Asparagine | 100 |
| L-Arginine | 50 |
| L-Alanine | 20 |
| L-Serine | 20 |
| L-Glutamine | 1000 |
| Myo-Inositol | 1000 |
| Maltose | 25,000 |
| Glucose | 10,000 |
| PEG 8000 | 120,000 |
| Activated Charcoal | 1000 |

Rinse media is the same as DM only without the glucose, PEG 8000, and activated charcoal added.

### Example 2

### Treatments

In this experiment, additional sugars: galactose, trehalose, and palatinose, and additional sugar combinations and concentrations will be tested and compared to BM, which has a 3% maltose concentration. Trehalose and palatinose are assumed to be unmetabolizable, and thus will be combined with lower concentrations of maltose to raise the osmolality back to that of BM. Equal concentrations of galactose and glucose will be combined and tested.

The effect of low sugar concentration under fed-batch conditions is unknown and may differ significantly from results determined for Erlenmeyer shake flasks. Sugar concentration (Brix) is regulated through feedback control on the Wave bioreactor platform, and thus it does not allow complete exhaustion of sugar. Should the sugar concentration drop below target (“setpoint”), the Brix feedback controller will increase media addition to drive the sugar concentration back to setpoint.

Each genotype was bulked so that the time between cryo retrieval and application of the treatments closely matches the typical period of time in the production system between cryo retrieval and first harvest. Emphasis was placed on the treatment effects during the earliest periods in the Wave bioreactors as it was anticipated that a commercial production facility will attempt to quickly bulk each genotype and then only plate it one or two times, as opposed to the current operation where each culture is available to plate for 3 months or more. Ten or more cryo vials will be retrieved for each genotype and bulking will be done as soon as possible in 1 L and/or 5 L Cellbags.

Ten vials of each genotype were removed from cryo storage and each multiplication plate received 2 to 3 vials. Once there was sufficient growth, the cultures were transferred off filter paper and to fresh solid multiplication plates and cultured using 3× hormone at 25° C. Once there were sufficient cells, they were transferred to a Erlenmeyer shake flask and bulked until there was more than 55 ml of SCV in a 1 L Erlenmeyer shake flask. Flasks were shaken on the large shaker and cultured at 20° C. If the Wave bioreactor was ready to receive the culture, it was bulked in a 1 L Cellbag and then in a 5 L Cellbag until there was a total of approximately 4 L of culture. If the Wave bioreactor was not ready to receive the culture, it continued to bulk in 1 L flasks, and then the flasks were transferred to a 5 L Cellbag, where again the culture will be bulked to approximately 4 liters. All bulking on the Wave bioreactor was done at 23° C. and dissolved oxygen (“dO”)=50% of saturation.

Once bulking was complete, a sufficient volume of culture was aliquoted to a large glass container with stir bar, one per treatment. The total volume (or mass) of culture was noted, and all spent control media was completely aspirated. Each treatment media was added to return the culture to its original volume (or mass), this included the control treatment. This resulted in a sudden change in osmolality, but this procedure immediately replaced almost all of the control media with the treatment media.

Eight different media (treatments) were considered in this experiment. All media was based on BM with the reported sugar(s) and sugar concentration(s) in Table 3. Treatment 1 was Media BM. Each treatment was randomly assigned to a bioreactor on each of the Wave bioreactor units, thus there were two experimental units per treatment. Table 3 also indicates the mean Brix and Osmolality for all batches and bags of media.

Table 3. Sugar Type and Concentration for Each Media (“Treatment”) with Brix and Osmolality Indicated

TABLE 3

| | | | | | | Osmo- |
|---|---|---|---|---|---|---|
| Treat- | Malt- | ½ Glucose + | Treha- | | Brix, | lality, |
| ment | ose | ½ Galactose | lose | Palatinose | % | mmol/kg |
| 1 | 3.0% | | | | 3.20 | 126* |
| 2 | | 1.5% | | | 1.85 | 124 |
| 3 | 1.0% | | | | 1.33 | 73 |
| 4 | | 0.6% | | | 0.97 | 77 |
| 5 | 1.0% | | 2.0% | | 3.10 | 123 |
| 6 | 1.0% | | | 2.0% | 3.12 | 124 |
| 7 | 0.6% | | | | 0.95 | 64 |
| 8 | 0.6% | | | 2.4% | 3.12 | 122 |

*Based on Production data for BM with similar Brix (3.24%) - Many batches and bags were averaged together

All treatments were performed under the following conditions:
Target Density=2.5 g/L (via Brix feedback control)
Target Delta Brix=0.4% (e.g. Media Brix=3.2%=>Target Brix=2.8%)
Subculture interval=1 week
Inoculation volume=80 ml to 500 ml (dependent on the growth rate)

### Plating

Cells were plated based on capacitance, and a capacitance of 4.0 pF for the plating mix was the target. Cells were harvested, capacitance was checked by using 2 aliquots to create a mean number for the culture, and rinse media was added in the appropriate amount to reach the 4.0 pF target. Otherwise, cells were plated to semi solid medium or liquid development media. Specifically, plating was accomplished a plating method that is generally used to move pre-embryo suspensions through a development phase to maturation. The word ‘plated’ will be used to describe cells being applied to any development medium and it should be understood that the medium may be in a plate or some other vessel. Before plating, settled ESM must first be diluted to allow for easier pipetting and better distribution on the nylon surface. Rinse media, used as diluent, and ESM are placed into previously autoclaved, stir-bar containing, cytostir beakers. It is important to make sure that there is enough room at the top of a full beaker for the ESM to stir properly. There must be enough ESM in the beaker to cover the stir bar during plating so the stir bar will not hit the pipette tip. Flame the side arm(s) of the cytostir beakers before using; taking care to avoid melting the plastic lid, because once cells are added the beakers need to be kept upright to prevent them from leaking. Index the rinse medium before starting. Settle, index and mark the settled cell line (as if doing a liquid transfer) on the flask side. Samples for observation of ESM quality may be taken at any point. Aspirate supernatant (using hose with back flow valve). If using cells to continue in multiplication, transfer appropriate volume of settled cells to a fresh multiplication flask first. Then, mix and transfer the desired volume of SCV to be plated to appropriate size of cytostir flask (see table above). Loblolly pine ESM should be “rinsed” of auxins and cytokinins using a rinse media. To do this, add an equal amount of rinse medium to the cytostir vessel. Mix gently using the stir bar on stir plate. Auxins and cytokinins will be leached into the rinse media and when plated the rinse media will be removed. Plate ESM as soon as possible once prepared. The amount of time cells are allowed to remain off of the shaker should be limited since cells that have been standing for more than two hours have been found to have reduced growth potential after plating. Cytostir vessels with cells and rinse can be placed onto stir plates and left spinning for up to four hours.

Place the cyto-stir beaker, containing ESM mixture, on a stir plate and choose a stirring speed that will allow the entire volume of the beaker to be gently rotating; there should be neither damage to nor settling of ESM. Reduce the speed as needed as the volume goes down. Place sterile vacuum plating system (VPS) unit into hood.

Open autoclaved 2″×2″ nylon squares (for plates) or D-frame (for boxes). Use sterile forceps to move the nylon squares (up to 6 at a time) to the sterile VPS unit in the hood. Or use long forceps, D-frame tool of choice or hemostats to move a D frame to the sterile VPS unit in the hood. Use either a 30 ml glass pipette or Rainin pipettor to dispense cells. If using a Rainin pipettor, dispense cells in ‘drop’ fashion. Remove a Rainin pipettor from its crisper, adjust volume to that desired, and put on a pre cut, plugged and sterilized tip. When resetting the Use a sterile Petri plate, one for each cytostir, to dispose of the cells in the tip at the end of the cycle. The standard plating volume has been set to 1.0 ml for Loblolly pine cultures in plates. This is a one to one ratio of settled cells to rinse. Use 4 mls of cells diluted with 46 mls of rinse for D-frames. Remove the screw cap from the side arm of the cyto-stir beaker and set it aside. Slowly and carefully withdraw an aliquot of the ESM mixture into the pipette or pipettor tip. Dispense the aliquot onto each of the membranes or onto the surface of the D-frame. The rinse should allow for aliquots to spread evenly over the surface of the membrane. Change pipette/pipette tips between genotypes, flasks, and cyto-stirs. Turn on the VPS unit to vacuum away the rinse media. Move the nylon squares or the frame to media in appropriate vessel. Wrap finished plates with Parafilm (boxes require 2″ wide, ≈9″ long) using long strips twice around. Store the plates in clean crispers without lids, or boxes, on shelves in the dark. Cover with sheets of foil to keep cultures dark while allowing air to circulate. Leave in the dark for the development period.

Three ⅓ boxes per bioreactor were created. Each bioreactor was plated 3, 5, and 7 weeks following the date they started on the treatment media. Note that the culture Brix value for all 8 treatments started very near to their media Brix value due to the complete aspiration of the bulking (control) media, and thus culture Brix needed to decrease nearly 0.4% to reach set point. This transition took approximately 2 weeks if the weekly growth was doubling four times per week. The transition was shorter or longer dependent on the growth rate.

Development media was made just in time for the first plating to allow for the maximal time for recurring platings to happen using the same batch of media if possible. Media needs for each plating were 48×400 mls (19,200 mls). The media was split up into 3 batches of 9 L through the media clave from one basal media batch.

Assessment of the boxes occurred at 12 weeks of development. A cursory assessment was done from 5 to 8 weeks after plating to note morphologies.

Selection Criteria for Embryos:

Symmetrical embryos without obvious defects that have four or more cotyledons without any fused cots or cots sprouting from the center were chosen. Embryos had all three parts: cotyledons, hypocotyl, and radical regions. Sizes varied. A slight curve to the hypocotyl region was acceptable. Embryos with split radical regions were not selected.

Embryos were generally opaque and acceptable colors were shades of white, yellow or green. No translucent embryos, or vitrified green embryos were selected.

### Statistical Analysis

This experiment is a randomized complete block design. The blocks are the plating times and the treatments different sugars and concentrations. The treatments are detailed in Table 3. There are four genotypes that were plated 3 times.

The responses analyzed for this report are the weekly growth, embryos per d-frame, category 1 germination, and for category 1+2: germination, germinants per d-frame, root length, the hypocotyl length, and the epicotyl length.

The proportion responses are analyzed using a generalized linear model with a logit link and binomial distribution after categorizing as a 0/1 response. The continuous responses were analyzed with a mixed linear model after taking the log transformation to account for increasing variability with higher means. The means and confidence intervals are back transformed to the original scale. By using the log transformation, the weight of star performing treatments/genotypes on the mean estimates were reduced.

The analysis is presented in a series of tables. The mean estimate for each treatment is reported. The column “Test at α=0.10” summarizes test results comparing combined means. Means with the same symbol are not statistically different at α=0.10.

### Multiplication

The usual suite of multiplication QC measures, e.g., Brix, culture density, etc., are reported here. Morphological observations were made weekly. Palatinose based treatments were the only cultures to clearly stand out from the other treatments. They generally became more organized and synchronized after several weeks of treatment, but they were also brown in color, and became very brown and less organized by the end of the treatment.

### Brix

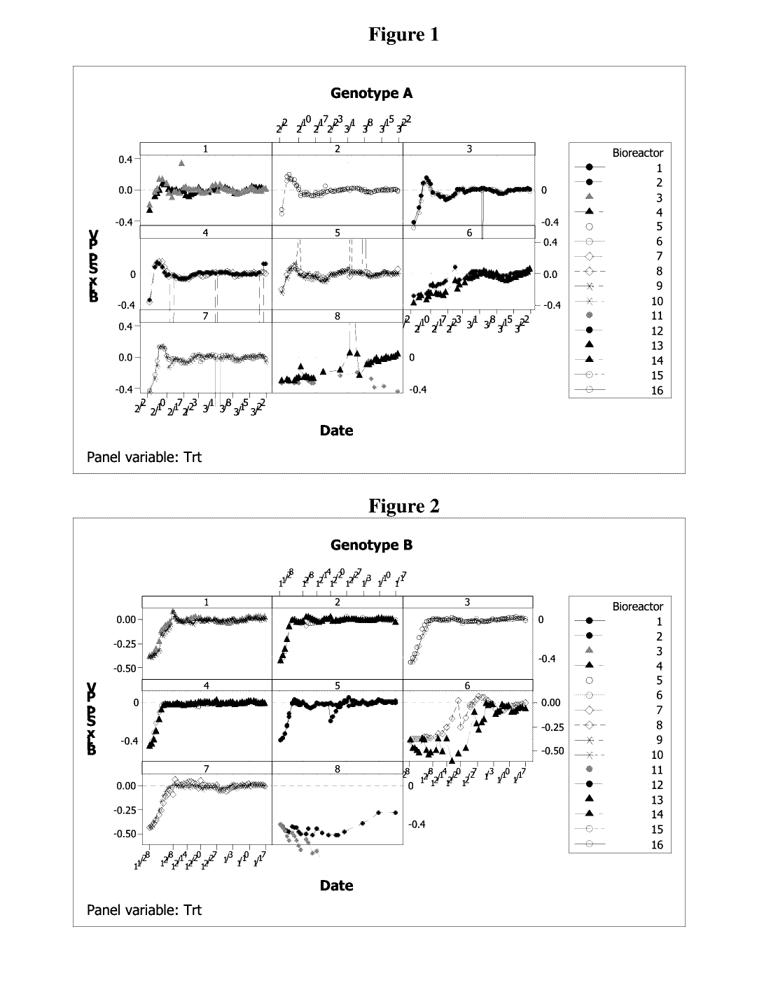

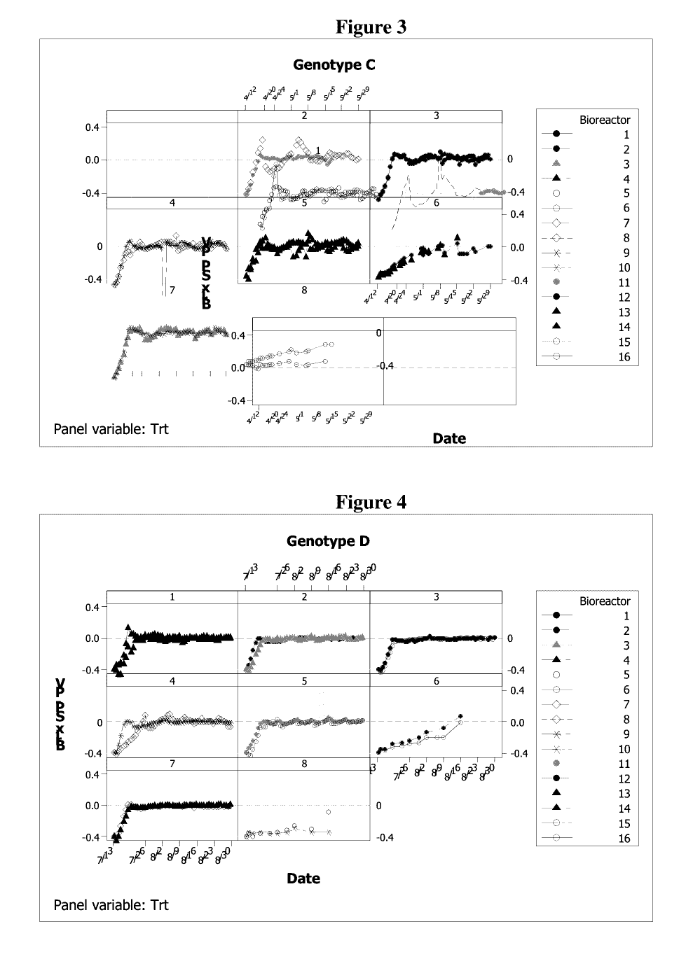

FIGS. 1-4 show the trends for the Brix Setpoint (“SP”) minus the Brix measurement (“PV”). The data is presented in this way as most treatments were controlled at significantly different Brix values. In each figure, the panel variable is treatment, and each figure shows data for a different genotype. Brix is at setpoint when SP-PV equals zero.

To initiate treatment, all control media was completely aspirated, and an equivalent volume of treatment media was added. This resulted in all treatments starting at SP-PV approximately equal to −0.4%, i.e., at the Brix of the treatment media. The initial media feed rate was typically zero and the Brix feedback controller was turned on. Brix can only drop (i.e., SP-PV can only increase) if there is growth; the Brix feedback controller can only add media, it cannot take sugar away. No growth is indicated by SP-PV remaining at −0.4% or decreasing, and slow growth is indicated by a large period of time being needed for SP-PV to first reach zero.

FIGS. 1-4 show that only 1 of 8 cultures for Treatment 8 consumed enough sugar to reach SP-PV=0. This is indicative of no to extremely slow growth. The figures also show that all cultures of Treatment 6, also with palatinose, were able to reach SP-PV=0, albeit much slower than the cultures without palatinose. This shows that Treatment 6 grew significantly slower than the treatments without palatinose. It is demonstrated that simply exchanging 0.4% palatinose for 0.4% maltose (i.e., T8->T6), made the difference between no growth and some growth, suggesting that the ratio of palatinose to maltose is important.

For the non-palatinose based treatments (i.e., those that grew at any appreciable rate) Brix control was excellent. A closer examination of FIGS. 1-4 show that control was slightly better for some genotypes. As will be shown later, these genotypes grew slower than other genotypes.

### Osmolality

A factor believed to be important in liquid multiplication is osmolality. The experimental treatments were designed to control for it. Treatment 3 has only 1% maltose, while Treatments 5 and 6 also have 1% maltose, with 2% of a non-metabolizable sugar, Trehalose and Palatinose, respectively. Treatment 7 has 0.6% maltose, while Treatment 8 has 0.6% maltose and 2.4% Palatinose.

Table 4 shows the mean osmolalities. Any measurements taken during the startup phase, i.e., before first harvest (approximately before 2 weeks of culturing) were not included. Treatments 6 and 8 were designed to have about the same osmolality as Treatments 1, 2, and 5, but they were higher due to their slow consumption of sugar. Treatment 8 with its very limited sugar consumption had an osmolality that was approximately equal to that of the media. Treatment 7 had the lowest osmolality as expected. Such a low osmolality was expected to be detrimental to culture growth and quality.

TABLE 4
Mean Osmolality, mmol/kg

| Treatment | Mean | N |
|---|---|---|
| 8 | 128 | 11 |
| 6 | 111 | 15 |
| 1 | 103 | 25 |
| 5 | 103 | 25 |
| 2 | 96 | 25 |
| 3 | 51 | 25 |
| 4 | 47 | 25 |
| 7 | 41 | 25 |

### Weekly Growth

There are no obvious trends in the data as the variation looks fairly random. A significant portion of the variation is from measurement error. The estimated 95% confidence interval for a weekly growth measure is +/−0.80 times at 5 times and +1-1.6 times at 10 times. Note too that measurement error will cause weekly cycles in the data as the harvest dry weight measurement is the same as the inoculum dry weight measure for the next fed-batch. Thus a high measurement one week, will lead to a high growth estimate, but the following week it, will depress the growth estimate.

Table 5 shows the treatment least square means (“lsmeans”) and pairwise comparisons from the statistical analysis for weekly growth relative to Treatment 1, i.e., standard protocol. All relative variables were determined by dividing the response of a treatment by the response for Treatment 1. There is significant evidence of a treatment effect (p-value <0.0001). Note that Treatment 8 was not included in the statistical analysis as it did not grow. Also, the reported mean value for Treatment 6 is for when it was growing, which was generally during the second half of the treatment phase.

As the Brix data indicated, Treatment 8 did not grow, while Treatment 6 was slow to start growing and when it did, it grew slowly. The small exchange of 0.4% palatinose for 0.4% maltose made the difference between no growth and some growth. Such sensitivity to palatinose or possibly the ratio of palatinose to maltose warrants further investigation especially given the germination results. Palatinose was the only sugar type that had an impact of growth. Trehalose and Galactose+Glucose did not.

Treatment 7 grew the fastest, and this is most likely due to an osmotic effect. But note that Treatments 3 and 4, which only had slightly higher osmolalites than Treatment 7, grew slower and at approximately the same rate as control. Thus this osmotic effect is only significant at very low osmolality, i.e., very low maltose concentrations.

What is surprising is how well cultures can grow on little excess sugar. Both Treatments 4 and 7 only had about 0.2% excess sugar, as a sugar concentration was 0.4% below media Brix. This will result in a low sugar gradient between the media and the cells, but it did not hinder growth in liquid multiplication. Maltose concentrations could be reduced to 1.0% without affecting growth. Another reason to reduce its concentration is so that other compounds could be added without changing the osmolality, compounds such as a pH buffer.

TABLE 5
Relative Mean Weekly Growth, x

| | Sugar, % | | | | | |
|---|---|---|---|---|---|---|
| Treat- | | GG | | | | Test at |
| ment | MM | G | TT | PP | Mean | α = 0.10 |
| 7 | .06 | | | | 1.34 | A |
| 2 | | 1.5 | | | 1.11 | B |
| 3 | 1.0 | | | | 1.05 | B |
| 5 | 1.0 | | 2.0 | | 1.03 | B |
| 4 | | .06 | | | 1.03 | B |
| 1 | 3.0 | | | | 1.00 | B |
| 6 | 1.0 | | | 2.0 | 0.37 | C |
| 8 | 1.6 | | | 2.4 | 0.26 | N/A |

Table 6 shows the treatment lsmeans and pairwise comparisons from the statistical analysis. There is no evidence of a treatment effect (p-value=0.40). This result is surprising given how poorly Treatments 6 and 8 grew in liquid multiplication. The results for Treatment 8 do have a limited inference base as two genotypes dropped out of this study for it, but Treatment 6 included results for all genotypes and up to all 3 platings.

TABLE 6
Relative Mean Embryos Per d-frame

| | | Test at |
|---|---|---|
| Treatment | Mean | α = 0.10 |
| 1 | 1.00 | A |
| 5 | 0.95 | A |
| 7 | 0.88 | A |
| 8 | 0.86 | A |
| 4 | 0.85 | A |
| 2 | 0.84 | A |
| 3 | 0.83 | A |
| 6 | 0.71 | A |

### Germination

Table 7 shows the treatment lsmeans and pairwise comparisons from the statistical analysis. There is strong evidence of a treatment effect (p-value=0.0003).

Table 7 clearly shows that sugar type was a significant factor while sugar concentration was not. The palatinose treatments had significantly higher germination than the other treatments, and the galactose+glucose treatments had significantly lower germination. There is no evidence that adding trehalose had an effect on germination.

While the differences seem small from a processing/economical perspective, bear in mind that the reported lsmeans are for data transformed back from the log scale. This analysis approach will reduce the weight of star performing treatments/genotypes on the lsmeans. In addition, the true mean is unknown and lies somewhere between the lower and upper limits with 90% confidence.

TABLE 7
Relative Category 1 Germination

| | Sugar, % | | | | | |
|---|---|---|---|---|---|---|
| Treat- | | GG | | | | Test at |
| ment | MM | G | TT | PP | Mean | α = 0.10 |
| 8 | 0.6 | | | 2.07 | 2.07 | A |
| 6 | 1.0 | | | 1.55 | 1.55 | A |
| 7 | 0.6 | | | 1.16 | 1.16 | B |
| 5 | 1.0 | | 2.0 | 1.14 | 1.14 | BC |
| 3 | 1.0 | | | 1.11 | 1.11 | BC |
| 1 | 3.0 | | | 1.00 | 1.00 | BCD |
| 4 | | 0.6 | | 0.89 | 0.89 | CD |
| 2 | | 1.5 | | 0.86 | 0.86 | D |

Table 8 shows the treatment lsmeans and pairwise comparisons from the statistical analysis. There is strong evidence of a treatment effect (p-value=0.002).

TABLE 8
Relative Category 1 + 2 Germination

| | Sugar, % | | | | | |
|---|---|---|---|---|---|---|
| Treat- | | GG | | | | Test at |
| ment | MM | G | TT | PP | Mean | \grave{α} = 0.10 |
| 8 | 0.6 | | | 2.4 | 1.48 | A |
| 6 | 1.0 | | | 2.0 | 1.44 | A |
| 3 | 1.0 | | | | 1.11 | B |
| 7 | 0.6 | | | | 1.11 | B |
| 5 | 1.0 | | 2.0 | | 1.10 | B |
| 2 | | 1.5 | | | 1.01 | B |
| 1 | 3.0 | | | | 1.00 | B |
| 4 | | 0.6 | | | 0.95 | B |

Table 9 also shows that sugar type was a significant factor while sugar concentration was not. For Category 1+2 germination, the palatinose treatments were significantly better than the control treatment. No statistically significant differences were detected between the non-palatinose treatments.

TABLE 9
Relative Root Length

| | | Test at |
|---|---|---|
| Treatment | Mean | \grave{α} = 0.10 |
| 2 | 1.10 | A |
| 3 | 1.03 | A |
| 5 | 1.03 | A |
| 1 | 1.00 | A |
| 4 | 0.99 | A |
| 6 | 0.97 | A |
| 7 | 0.96 | A |
| 8 | 0.94 | A |

TABLE 10
Shows the Treatment Ismeans and Pairwise Comparisons From the
Statistical Analysis. There is No Evidence of a Treatment Effect
(p-value = 0.35).

| | | Test at |
|---|---|---|
| Treatment | Mean | \grave{α} = 0.10 |
| 1 | 1.00 | A |
| 2 | 1.00 | A |
| 5 | 1.00 | A |
| 3 | 1.00 | A |
| 7 | 1.00 | A |
| 4 | 0.98 | A |
| 6 | 0.95 | A |
| 8 | 0.93 | A |

Sugar type was the dominant factor in this study. Sugar concentration only resulted in a modest effect on one response variable, liquid multiplication growth rate. The responses to palatinose were very intriguing. Only a slight increase in its concentration from 2.0% (T6) to 2.4% (T8) (and equivalent reduction in maltose) arrested growth in liquid multiplication. Neither treatment is a viable liquid multiplication treatment due to their lack of significant growth rate. It is possible that palatinose interferes with maltose metabolism, and thus the ESM is starving. Another experiment demonstrates that 2.4% palatinose+0.3% glucose grew well in liquid multiplication, while 2.4% palatinose+0.6% maltose (T8) did not grow. The ratio of palatinose to maltose rather than the absolute concentration of palatinose is likely a factor affecting the growth rate. Palatinose resulted in a significant increase in germination compared to all other treatments.

The beneficial properties of palatinose are not limited to multiplication. The addition of palatinose to the synthetic gametophyte of manufactured seed has benefits for root initiation. As the palatinose concentration increased from 0 to 40 g/l in 20 g/l increments, root presence improved. This is true when the base sugar was sucrose or glucose.

The Palatinose treatments can be used as pre-treatments and the duration of the pre-treatment can be varied. In addition, lowering the concentration of palatinose relative to maltose may allow acceptable growth rates in liquid multiplication and still result in a germination improvement.

Trehalose, which is non-metabolizable sugar like palatinose, had no significant effect on any response variable relative to the control treatment. The treatments with Galactose-Glucose resulted in the lowest germination; statistically significant for Category 1 relative to all treatments except for the control treatment.

### Example 3

### Methods

This experiment is a randomized complete block design. The plating is the block. There are 4 treatments in a one-way layout that are detailed in Table 11.

| Trt # | Description |
|---|---|
| 1 | 3.0% Maltose |
| 2 | 1.5% Glucose |
| 3 | 0.6% Maltose and 2.4% |
| | Palatinose |
| 4 | 0.3% Glucose 2.4% Palatinose |

The responses considered for these analyses are:

Cat. 1 Organ Lengths: Root, Hypocotyl, & Epicotyl Cat. 1 Germ %

Cat. 1+2 Germ %

Number of Cat. 1 per plate

Number of Cat. 1+2 per plate

Yield

Proportion responses were modeled using a generalized linear model with the binomial distribution and logit link. The continuous responses were analyzed with linear models. The count responses were analyzed using a generalized linear model with the Poisson distribution and log link. Treatment means and confidence intervals were transformed back to the natural scale for all responses.

### Root Length

Treatment lsmeans in a relative scale are given in Table 12,

TABLE 12
The Relative Means and Confidence Intervals for Treatments

| | | Test at |
|---|---|---|
| Treatment | Mean | \grave{α} = 0.10 |
| 1 | 1.00 | A |
| 2 | 0.82 | A |
| 3 | 0.63 | A |
| 4 | 0.97 | A |

Table 12: Mean relative root length in the original scale. The column “Test at α=0.10” summarizes test results comparing combined means. Means with the same symbol are not statistically different at =0.10.

### Hypocotyl Length

The different symbols show the different bioreactors. There is no evidence of a treatment effect (p-value=0.37). Treatment lsmeans in the relative scale are given in Table 13.

TABLE 13
Mean Hypocotyl Length in the Relative Scale

| | | Test at |
|---|---|---|
| Treatment | Mean | \grave{α} = 0.10 |
| 1 | 1.00 | A |
| 2 | 1.05 | A |
| 3 | 0.95 | A |
| 4 | 1.07 | A |

### Epicotyl Length

There is no evidence of a treatment effect (p-value=0.54). Treatment lsmeans in the relative scale are given in Table 14.

TABLE 14
Mean Epicotyl Length in the Relative Scale

| | | Test at |
|---|---|---|
| Treatment | Mean | \grave{α} = 0.10 |
| 1 | 1.00 | A |
| 2 | 0.88 | A |
| 3 | 0.99 | A |
| 4 | 0.88 | A |

### Category 1 Germination Percentage

There is moderate evidence of a treatment effect (p-value=0.04). Treatment lsmeans are given in Table 15.

TABLE 15
Mean Relative Cat 1 Germination

| | | Test at |
|---|---|---|
| Treatment | Mean | \grave{α} = 0.10 |
| 1 | 1.00 | A |
| 2 | 0.74 | A |
| 3 | 0.87 | A |
| 4 | 0.26 | B |

### Category 1+2 Germination Percentage

There is no evidence of a treatment effect (p-value=0.81). Treatment lsmeans are given in Table 16.

TABLE 16
Mean Relative Category 1 + 2 Germination

| | | Test at |
|---|---|---|
| Treatment | Mean | \grave{α} = 0.10 |
| 1 | 1.00 | A |
| 2 | 0.75 | A |
| 3 | 0.88 | A |
| 4 | 0.75 | A |

### Category 1 Germination Count

There is moderate evidence of a treatment effect (p-value=0.06). Treatment lsmeans are given in Table 17.

TABLE 17
Mean Relative Category 1 Germination Count

| | | Test at |
|---|---|---|
| Treatment | Mean | \grave{α} = 0.10 |
| 1 | 1.00 | A |
| 2 | 0.73 | A |
| 3 | 0.86 | A |
| 4 | 0.27 | B |

### Category 1+2 Germination Count

There is moderate evidence of a treatment effect (p-value=0.84). Treatment lsmeans are given in Table 18.

TABLE 18
Mean Relative Cat 1 + 2 Germination Count

| | | Test at |
|---|---|---|
| Treatment | Mean | \grave{α} = 0.10 |
| 1 | 1.00 | A |
| 2 | 0.78 | A |
| 3 | 0.83 | A |
| 4 | 0.72 | A |

Treatment lsmeans are given in Table 19.

TABLE 19
Mean Relative Yield

| | | Test at |
|---|---|---|
| Treatment | Mean | \grave{α} = 0.10 |
| 1 | 1.00 | A |
| 2 | 0.98 | A |
| 3 | 1.61 | B |
| 4 | 1.16 | A |

### Example 4

### Methods

This experiment is a randomized complete block design. The genotypes are the blocks. There are 4 treatments detailed in Table 20. There are 3 genotypes. Each treatment is applied to two bioreactors per genotype. There are 3 plating occasions for genotype.

TABLE 20
Treatments

| | |
|---|---|
| 1 | Control (3.0% maltose). |
| 2 | 3.0% Palatinose |
| 3 | 0.6% Maltose + 2.4% Palatinose |
| 4 | 0.3% Glucose + 2.4% |
| | Palatinose |

The responses considered for this analysis are category 1 germination and root length. Germination is analyzed with a generalized linear model with a binomial distribution and logit link after summing up to the experimental unit level. Root length is analyzed with a linear model after averaging root lengths to the experimental unit level. Means are back transformed to the natural scale for plots and tables.

### Category 1 Germination

There is no evidence of a plating*treatment effect (p-value=0.23). There is suggestive evidence of treatment effect (p-value=0.09). There is moderate evidence of a weak effect (p-value=0.07). There is strong evidence of a genotype effect (p-value <0.0001). Treatment lsmeans and comparisons are given in Table 21. Treatment*week lsmeans are given in Table 21.

TABLE 21
Relative category 1 germination by treatment. The column “Test at
α = 0.10” summarizes test results comparing combined means.
Means with the same symbol are not statistically different at α = 0.10.

| | | Test at |
|---|---|---|
| Treatment | Mean | α = 0.10 |
| 1 | 1.00 | B |
| 2 | 1.43 | A |
| 3 | 1.19 | AB |
| 4 | 1.08 | B |

Tables 22, 23, and 24: Relative Category 1 Germination By Treatment×Plating

TABLE 22
Plating 1

| Treatment | Mean |
|---|---|
| 1 | 1.00 |
| 2 | 0.98 |
| 3 | 1.02 |
| 4 | 0.71 |

TABLE 23
Plating 2

| Treatment | Mean |
|---|---|
| 1 | 1.00 |
| 2 | 1.88 |
| 3 | 1.12 |
| 4 | 1.15 |

TABLE 24
Plating 3

| Treatment | Mean |
|---|---|
| 1 | 1.00 |
| 2 | 1.54 |
| 3 | 1.46 |
| 4 | 1.54 |

### Root Length

There is no evidence of a plating*treatment effect (p-value=0.94). There is moderate evidence of treatment effect (p-value=0.02). There is strong evidence of a week effect (p-value=0.01). There is strong evidence of a genotype effect (p-value <0.0001). Treatment lsmeans and comparisons are given in Table 25. Treatment*week lsmeans are given in Table 25.

Table 25: Root length by treatment. The column “Test at α=0.10” summarizes test results comparing combined means. Means with the same symbol are not statistically different at α=0.10.

Tables 25, 26 (Plating 1), 27 (Plating 2), and 28 (Plating 3): Root length by treatment times week.

TABLE 25

| | | Test at |
|---|---|---|
| Treatment | Mean | \grave{α} = 0.10 |
| 1 | 1.00 | B |
| 2 | 1.15 | A |
| 3 | 1.15 | A |
| 4 | 1.08 | AB |

TABLE 26

| Treatment | Mean |
|---|---|
| 1 | 1.00 |
| 2 | 1.11 |
| 3 | 1.13 |
| 4 | 1.06 |

TABLE 27

| Treatment | Mean |
|---|---|
| 1 | 1.00 |
| 2 | 1.09 |
| 3 | 1.15 |
| 4 | 1.04 |

TABLE 28

| Treatment | Mean |
|---|---|
| 1 | 1.00 |
| 2 | 1.24 |
| 3 | 1.15 |
| 4 | 1.15 |

I/We claim:

1. A method of improving germination of plant embryos, comprising treating a culture of plant tissue with a multiplication medium comprising a plant non-metabolizable sugar.

2. The method of claim 1, wherein the plant non-metabolizable sugar is isomaltulose.

3. The method of claim 1, further comprising a metabolizable sugar.

4. The method of claim 3, wherein the ratio of non-metabolizable sugar to metabolizable sugar is 2:1 or 4:1.

5. The method of claim 3 wherein the metabolizable sugar is maltose.

6. The method of claim 1, wherein the plant tissue is a plant embryo.

7. The method of claim 6, wherein the culture consists of an embryo suspensor mass.

8. The method of claim 7, wherein the culture is treated for two weeks or less.

9. The method of claim 7, wherein the culture is treated for three weeks or less.

10. The method of claim 7, wherein the culture is a multiplication culture.

11. The method of claim 7, wherein the culture is earlier than a development culture.

12. The method of claim 1, wherein the liquid medium comprises 3.0% (w/w) isomaltulose.

13. The method of claim 1, wherein the liquid medium comprises 0.6% (w/w) maltose and 2.4% (w/w) isomaltulose.

14. The method of claim 1, wherein the liquid medium comprises 1.0% (w/w) maltose and 2.0% (w/w) isomaltulose.

15. The method of claim 1, wherein the liquid medium reduces cell growth of the culture.

16. The method of claim 1, wherein the liquid medium synchronizes the culture.

17. The method of claim 1, wherein category 1 germination frequency increases.

18. The method of claim 1, wherein category 1+2 germination frequency increases.

19. The method of claim 1, wherein the culture is maintained in a bioreactor.

20. The method of claim 19, wherein the culture is maintained in a flask.

21. The method of claim 20, wherein the culture is maintained in a shake flask under batch conditions.

22. The method of claim 19, wherein the culture is maintained in a Wave bioreactor platform bioreactor under fed-batch conditions.

23. A multiplication medium for liquid cultures of plant cells comprising isomaltulose.

24. The multiplication medium of claim 23, further comprising maltose.

25. The multiplication medium of claim 24, wherein the maltose is at a concentration of less than 3 g/l.
